(19) United States
(12) Patent Application Publication
(10) Pub. No.: US 2025/0082799 A1
(43) Pub. Date: Mar. 13, 2025

(54) A PORTABLE SYSTEM COMPRISING AN ULTRAVIOLET LIGHTING ARRANGEMENT

(71) Applicant: PUREFIZE TECHNOLOGIES AB, Uppsala (SE)

(72) Inventors: Jonas TIRÉN, UPPSALA (SE); Fredrik FORSSELL, HELSINGBORG (SE)

(73) Assignee: PUREFIZE TECHNOLOGIES AB, Uppsala (SE)

(21) Appl. No.: 18/728,138

(22) PCT Filed: Jan. 16, 2023
(86) PCT No.: PCT/SE2023/050040

§ 371 Date: Jul. 11, 2024

(30) Foreign Application Priority Data
Jan. 19, 2022 (SE) 2250041-7

(51) Int. Cl. A61L 2/10; A61L 2/24

(52) U.S. Cl. CPC A61L 2/10; A61L 2/24; A61L 2202/11; A61L 2202/121; A61L 2202/122; A61L 2202/14; A61L 2202/16

(57) ABSTRACT

The present invention generally relates to a portable system comprising an ultraviolet (UV) lighting arrangement, to for example be used in relation to reduce microorganisms and reducing transmission of pathogens in e.g. a hospitality environment.

### TECHNICAL FIELD

The present invention generally relates to a portable system comprising an ultraviolet (UV) lighting arrangement, to for example be used in relation to reduce microorganisms and reducing transmission of pathogens in e.g. a hospitality environment.

### BACKGROUND

Systems for disinfection of water, air, surfaces or certain equipment using ultraviolet (UV) light have been used for decades, at least. Some common applications have traditionally been water purification and disinfection of e.g., operation theaters. Recently the interest to use UV light in other applications have increased and has been further elevated by the ongoing pandemic of SARS-Cov2. The need to better prevent pathogens to spread between humans largely increased, not only to prevent and limit pandemics but to reduce spread of common disease, colds, stomach-flu, etc. For disinfection purposes, UV light typically within the wavelength range between 100-280 nm is used, defined as UVC light. UVC light have traditionally been generated using low pressure mercury lamps (LP-Hg lamps), predominantly emitting wavelengths around 254 nm. Medium and/or high-pressure Hg lamps may alternatively be used, for example in large systems such as for water disinfection as these lamps may deliver higher power output. These systems may be combined with particle filtering, reverse osmosis (for water disinfection) and other. The UVC lighting systems are popular since they do not use any chemicals (e.g. chlorine). However the use of mercury (Hg, as provided in relation to low, medium and high pressure mercury lamps) is in general banned since it is a very toxic element. This is driving the emergence of new technologies.

One such technology becoming popular are UVC LEDs. Such devices are significantly smaller and also delivers significantly less output power as compared to mercury lamps. Yet another emerging technology is the field emission based UVC technology. This technology also offers small devices (although larger than UVC LEDs) and output less power as compared to mercury lamps.

The UV disinfection technology has also found its use in other equipment that is frequently interacted with by a user, such as for example within refrigerators. For example, by introducing such UV disinfection technology it may be possible to prolong the freshness of fresh food products stored within the refrigerator. Similarly, the UV disinfection technology can potentially be used in relation to general storage device, where e.g. food items such as fruit and meat can be stored and easily accessible through a sliding door or similar.

An example of an exemplary refrigerated storage device is presented in U.S. Ser. No. 10/849,996. U.S. Ser. No. 10/849,996 suggests implementing a computer system that is configured to control a UV light source that is turned off when the sliding door is opened, and then again turned on when the sliding door has been closed. The solution as is presented in U.S. Ser. No. 10/849,996 suggest controlling a dose of ultraviolet radiation delivered by the UV light source to treat food items stored at a shelf of the refrigerated storage device with the intention to extend the shelf life of the food items.

The refrigerated storage device U.S. Ser. No. 10/849,996 presents an interesting approach to reducing any risks involved in consuming the stored food items. However, the suggested solution is not always practical, specifically in many real situations where it not desirable to move the items firstly into a container and subsequently remove them to be placed at the intended area or into the intended volume of use. In addition, the removal process will, especially if performed with human interaction, cause a new risk of contamination, occurring after the intended disinfection, rendering it possibly less affective to prevent spread of pathogens.

With the above in mind, there appears to be an opportunity to introduce further improvements, with an overall desire to minimize microorganisms not just in relation to refrigerated storage devices, generally also for other areas frequently interacted with by multiple different humans.

### SUMMARY

According to an aspect of the present disclosure, the above is at least partly alleviated by a portable system for reducing microorganisms, wherein the system comprises a housing arranged to receive items provided in relation to consumption of food, an ultraviolet (UV) lighting arrangement positioned within the housing and arranged to illuminate the items received within the housing, a driver adapted to provide a drive signal to the UV lighting arrangement, and a control unit arranged to estimate an amount of human interaction with an interior of the housing and to selectively activating the driver based on the estimated amount of human interaction.

The present disclosure is based upon the understanding that standardized dosage of UV lighting (measured in e.g. mJ/cm²) in relation to a storage device that is irregularly interacted with by humans is insufficient to guarantee that items within the storage device are not contaminated with pathogens, including bacteria and virus. The dosage needed is depending on the species of pathogens, and the required reduction of pathogens.

This is specifically apparent in a hospitality environment where it is hard to reliably predict exactly when, how and by whom the storage device will be interacted with. One option could be to “overdose” the items within the storage device with UV radiation. However, such a scheme would greatly reduce lifetime of a UV lighting arrangement comprised with such a storage device, and also result in an unwanted electrical cost of operation. UV radiation may also slowly decompose and deteriorate many polymers/plastic materials that may commonly be used in a hospitality environment, and thus it may additionally be beneficial to keep the UV dosage as low as possible.

The present inventors have contravened this dilemma by providing a system that is adapted to store items provided in relation to consumption of food, where the items are expected to be frequently and irregularly interacted with by humans, such as typically within a hospitality environment. The system has been specifically configured to estimate an amount of human interaction, estimate the possible impact of the human interaction and control the needed dosage of UV radiation based on the estimated impact of human interaction. By means of such a system it is made possible to exactly control the UV radiation dose to limit spread and contamination of pathogens, while at the same time maximizing lifetime of the UV lighting arrangement and minimizing other adverse effects. The resulting system will thus provide for a safer environment for the humans interacting with the stored items, at the same time as the cost of operation (of the system) is kept to a minimum.

A further advantage with the system according to the present disclosure lies in its portability, allowing the system to be readily moved between different usable areas. Such a portability is of course specifically useful in the above-mentioned hospitality environment, where general space used for human interaction may change throughout the day.

Accordingly, in a preferred embodiment at least a portion of a bottom of the housing is “open” allowing the housing to be easily positioned on top of any type of items, such as a plate with food items, a stack of plates, a collection of cutleries, a collection of napkins, etc. Thus, the portable system with the mentioned partial open bottom may be moved between different areas, e.g. in a restaurant like environment, to areas e.g. defined as “uncontrolled”, meaning that they are interacted with directly by visitors possibly without gloves may be expected to interact with the items stored within the housing, such as through an openable portion for allowing the human interaction with the items. In a preferred embodiment of the present disclosure the above mentioned the openable portion is automatically opened when a human is detected in a vicinity of the system, thereby reducing human touching the openable portion of the housing.

In a preferred embodiment of the present disclosure, the UV lighting arrangement comprises a plurality of UV light sources arranged spaced apart at a top section of the housing for achieving an UV light intensity uniformity at the items when the UV lighting arrangement is activated. The position of the plurality of UV light sources may in some embodiments be taken into account by the control unit, where possibly areas where the human interaction has been mostly focused can be dosed with an increased amount of UV light as possibly compared to areas where less human interaction has been estimated to have taken place.

The system preferably further comprises at least one sensor arranged in communication with the control unit, wherein information from the at least one sensor is acquired by the control unit for estimating the human interaction. Such a sensor may for example be selected from a group comprising a switch, a PIR sensor, a radar sensor, a reed switch, an opto electronic sensor, a pressure sensor. Other types of sensors are of course possible and within the scope of the present disclosure, including any form of sensors applying non-contact technology. It should also be noted that more than a single sensor may be used, where such a multitude of sensors may be to estimate where within the interior of the housing that the human interaction is estimated to have taken place.

In an embodiment of the present disclosure the UV lighting arrangement comprises a plurality of non-mercury based UV light sources. Possibly, such UV light sources may be configured to emit radiation within a wavelength range from around 210 nm to 350 nm. It is however preferred to allow the UV light sources to emit light having a wavelength interval including emission of UV radiation at 265 nm, being a possible peak value for germicidal effectiveness. In a preferred embodiment, a single light source (of the plurality of UV light sources) is adapted to emit UV radiation in a broader spectrum, i.e. covering both wavelength ranges using a single device.

In a preferred embodiment of the invention, the UV light sources comprise e.g. a plurality of UV(C)-LEDs and/or a combination of light sources based on different technologies to suit the application. The UVC-LEDs may additionally have several different wavelength peaks in order to better cover a specific wavelength range.

Furthermore, emerging technologies such as field emission light sources (FEL) may be used in relation to the present disclosure and offers turn on times that are in the order of milliseconds, mainly governed by the electronic drive unit. In comparison, Hg-LP lamps typically need a warmup time in the range of a few minutes before they will reach full output power. The fact that these light sources contain Mercury (Hg) which is very toxic further makes the use of these limited in cases of hospitality environments, especially near food. Alternative technologies recently emerging are UVC LEDs and UVC Field Emission Devices. Field emission light sources on the other hand may have lifetimes in the order of 1000-10000 hours depending on the desired power density and have been measured to reach efficiencies around 10%, albeit 4-5% in the UVC region.

An advantageous effect with using a field emission light source as the UV light source is that such a light source may be configured to emit UV light at a spectrum that is not a distinct peak around 254 nm but a more continuous spectrum in above mentioned range of 210-350 nm. Field emission UVC lamps have demonstrated the capability to continue the disinfection process and do not exhibit any significant tailing effect.

The field emission light source may in one embodiment comprise a field emission cathode and an electrically conductive anode structure. The field emission cathode typically comprises a plurality of nanostructures formed on a substrate, whereas the electrically conductive anode structure comprises a light converting material arranged to receive electrons from the cathode and to emit UV light. The light converting material may for example be selected to be at least one of LaPO4:Pr³⁺, LuPO3:Pr³⁺, Lu2Si2O7:Pr³⁺, YBO3:Pr³⁺, YAlO₃:Sc³⁺ or YPO4:Bi³⁺ or a similar light converting material. As an alternative, the light converting material may generally be seen as a phosphor material.

Preferably, the nanostructures preferably comprise at least one of ZnO nanostructures and carbon nanotubes. The plurality of ZnO nanostructures is adapted to have a length of at least 1 um. In another embodiment the nanostructures may advantageously have a length in the range of 3-50 μm and a diameter in the range of 5-300 nm.

Preferably, each the field emission light source is provided with a UV light permeable portion comprises at least one of Quartz, fused silica, UV transparent borosilicate and UV transparent soft glass. Such materials are suitable due to their inherent transparency to UV light.

Generally, a material/structure is considered to be “transparent” to ultraviolet light of a particular wavelength when the material/structure allows a significant amount of the ultraviolet radiation to pass there through. In an embodiment, the ultraviolet transparent structure is formed of a material and has a thickness, which allows at least ten percent of the ultraviolet radiation to pass there through.

During operation of the field emission light source, an in comparison high voltage is applied between the cathode and the anode. The electron energy used for consumer applications should be less than 10 kV and preferably less than 9 kV or soft X-rays generated by Bremsstrahlung will be able to escape the light source (it is otherwise absorbed by the anode glass). However, these levels are to some extent depending on glass thickness, thus higher voltages can be allowed if a thicker glass is used.

On the other hand. the electron energy must be high enough to effectively generate UV radiation. A preferred range for consumer applications is thus 4-9 kV and 7-15 kV for industrial applications (where some soft X-rays can be accepted).

The cathode and the anode are in in one embodiment arranged in an evacuated chamber, where the evacuated chamber is arranged under partial vacuum so that the electrons emitted from the cathode may transit to the anode with only a small number of collisions with gas molecules. Frequently the evacuated space may be evacuated to a pressure of less than 1×10⁻⁴ Torr.

The control unit is preferably adapted to deactivate the UV lighting arrangement upon human interaction with the system, since UV light may be harmful to the human eye and skin. The above mentioned at least one sensor may be used for achieving such a functionality. However, it may also be possible to arrange further sensors directed to an exterior of the housing, whereby the UV lighting arrangement is deactivated already when a human is in close vicinity of the system. Similarly, it is desirable to again activate the UV lighting arrangement upon the openable portion of the housing being detected to be in a closed state.

Yet another way to achieve prevention of the human eye or skin from UV light is to use a locking mechanism, the locking mechanism being activated by the control unit when the UV lighting arrangement is active.

The control unit is furthermore preferably arranged to control the driver such that a predetermined minimum amount of UV radiation is applied to the items over a predefined time period. The human interaction will as such form a “superimposed” activation of the UV lighting arrangement that is dependent on estimated human interaction.

In an exemplary embodiment it may be desirable to allow the estimated amount of human interaction to include a duration of the openable portion of the is opened. It may however also or instead be possible to estimate the amount of direct interaction with the items within the housing. Either or both of these estimations may then be used by the control unit to operate the driver to thereby emit UV towards the items within the housing. As indicated above, the activation of the driver based on the interaction may in some embodiments be superimposed, i.e. in addition to a baseline of UV radiation (per time unit).

In some embodiments the system may be arranged to further comprise a temperature sensor, where the temperature sensor in turn is arranged inside the housing. The temperature sensor is in such an embodiment connected to the control unit, whereby the control unit may be arranged to selectively activate the driver based on an average temperature within the housing as determined over a predefined time period. As an example, an in comparison higher temperature may result in an increased amount of UV radiation. The temperature may as such be used for defining the above-mentioned baseline of UV radiation.

Preferably, the control unit is further adapted to selectively activating the driver based on at least one of a distance to the items, a target micro-organism, or an expected user behavior in relation to the items. That is, if the system has been specifically configured to a specific type of operation, or with knowledge of a specific type of micro-organism, where E-coli and SARS-COV-2 are two examples, it may be possible to control a UV dosage and/or a target wavelength range of the UV light emitted by the UV lighting arrangement.

The drive unit may further be arranged to detect that the system has been moved, indicating that the items to be disinfected has not yet received a first disinfection UV dosage. In this case it may be desirable to use a larger initial dosage, whereas the subsequent doses, estimated by the impact of human interaction may be smaller. The same function may obviously be achieved by for example a button, where the user signals manually to the driver to give an initial, higher dosage.

In some embodiments the driver is connected to the mains for providing the necessary energy to operate the control unit and the UV lighting arrangement. It may however as an alternative or also be possible to provide the system with a battery or similar to provide the necessary energy. A state of charge of such a battery may correspondingly be taken into account when selectively activating the driver such that the UV lighting arrangement emits UV light. It may also, or alternatively, be possible to equip the system with at least one photovoltaic cell connected to the driver for delivering energy for powering the UV lighting arrangement. The photovoltaic cell may as such also be connected to the mentioned battery.

It may further be desirable to arrange the system to further comprise a reflective portion, where the reflective portion is adapted to increase the amount of UV light that is intended to be used for minimizing microorganisms in areas that could impact the wellbeing of e.g. humans. As such, rather than “escaping” the UV light is as such focused towards the surface that is to be treated.

As used herein, a material/structure is considered to be “reflective” to ultraviolet light of a particular wavelength when the material/structure has an ultraviolet reflection coefficient of at least thirty percent for the ultraviolet light of the particular wavelength. In a more particular embodiment, a highly ultraviolet reflective material/structure has an ultraviolet reflection coefficient of at least eighty percent.

Further features of, and advantages with, the present invention will become apparent when studying the appended claims and the following description. The skilled addressee realize that different features of the present invention may be combined to create embodiments other than those described in the following, without departing from the scope of the present invention.

### BRIEF DESCRIPTION OF THE DRAWINGS

The various aspects of the present disclosure, including its particular features and advantages, will be readily understood from the following detailed description and the accompanying drawings, in which:

FIG. 1 illustrates an embodiment of a portable system according to currently preferred embodiments of the invention,

FIGS. 2A and 2B shows exemplary implementations of the UV lighting arrangement comprised with the system as shown in FIG. 1,

FIGS. 3A-3B illustrates the emission spectra from an Hg light source and its corresponding germicidal de-activation curve,

FIGS. 4A-4F illustrates different emission spectra resulting from different phosphor material as used in relation to the present disclosure and their corresponding germicidal de-activation curves,

FIG. 5 illustrates the results from a mathematical model with and without a re-activation process, and

FIG. 6 provides diagrams showing estimated contamination within the housing and resulting operation of the UV lighting arrangement.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled addressee. Like reference characters refer to like elements throughout.

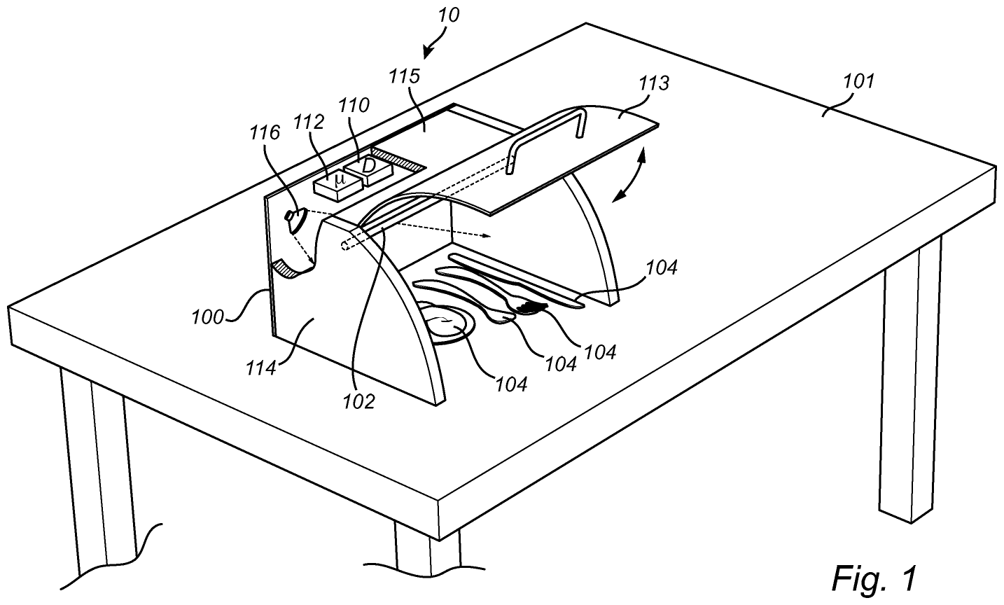

Referring now to the drawings and to FIG. 1 in particular, there is illustrated an embodiment of a portable system 10, comprising a housing 100. The system is in FIG. 1A placed onto a table/surface 101. Within an interior of the housing 100, there is arranged a UV lighting arrangement 102 configured to emit UV light towards a plurality of items 104 provided in relation to consumption of food. Such items may for example include a plate with food items, a stack of plates, a collection of cutleries, a collection of napkins, etc. The items 104 may for example be arranged at a bottom of the housing 100. In some embodiments, such as exemplified in FIG. 1, the housing 100 may be open, whereby some items instead will be arranged directly at e.g., the table 101 but still elsewhere “covered” by the housing 101. Optionally, the housing 100 may be provided with one or a plurality of internally arranged shelves (not shown). Preferably, such shelves are selected to be transparent to UV light.

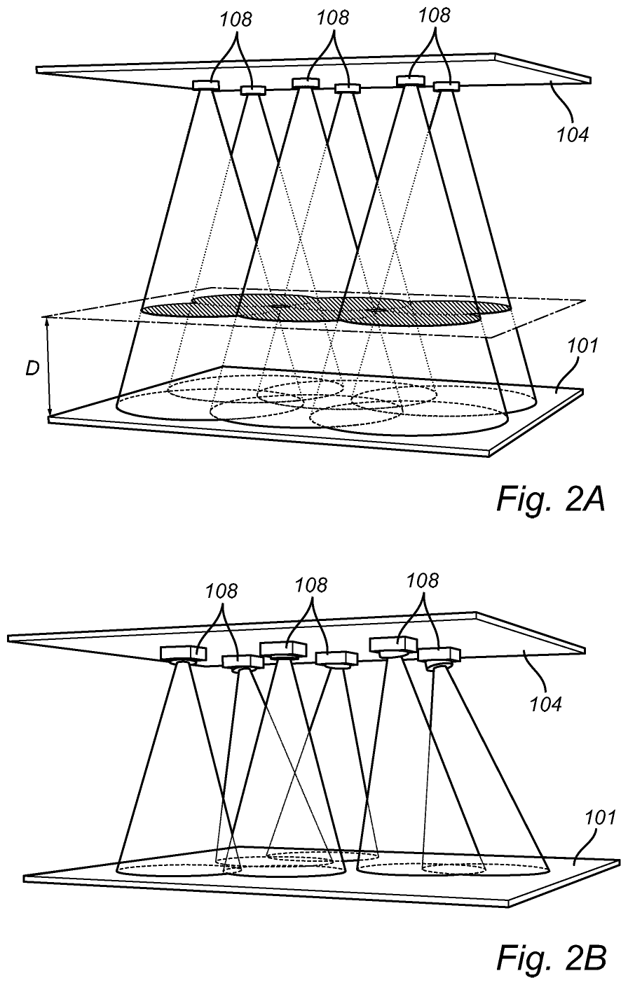

The UV lighting arrangement 102 in turn comprises a plurality of UV light sources 108 (as shown in FIGS. 2A and 2B). The UV light sources 108 are adapted to emit UV light within a predefined and possibly controlled wavelength range, where such a wavelength range preferably is between 210-350 nm. The UV light sources are preferably arranged so that the emitted UV light is directed so that optimum disinfection performance is reached at the area(s) deemed most relevant, for example where the highest impact of human interaction can be expected, or to yield an as uniform intensity as possible.

The system 10 further comprises a driver 110 connected to the UV lighting arrangement 102 and arranged to provide power for driving the UV light sources 108 of the UV lighting arrangement 104. The system 102 further comprises control unit 112, arranged in communication with the driver 110 and arranged to control the overall operation of the driver 110 for controlling the UV light sources 108 of the UV lighting arrangement 102. The control unit 112 and the driver 110 may be integrated into a single unit.

The system 10 further comprises at least one door 113 disposed on at least one side of housing 100 to allow access to an interior of the housing 100 and thus the items 104.

The housing 100 further comprises side walls 114 and top section 115. In some embodiments the side walls 114 and the top section 115 is provided with a UV-reflective material or coated with a UV-reflective material. However, in some embodiments the side walls 114 are transparent and allows for a user to “see” the items 104 within the housing 100 without having to open the door 113. In such an embodiment it may be desirable to provide the side walls 114 with a coating that is arranged to be opaque to UV light.

As exemplified in FIG. 1 and with further reference also to FIG. 2A, the plurality of UV light sources 108 of the UV lighting arrangement 104 are arranged at the top section 115 of the housing 100. In FIG. 2A six UV light sources 108 are provided, however any number of UV light sources 108 are possible and within the scope of the present disclosure. The exact number of UV light sources 108 may typically be dependent on the size of the housing 100, the expected number of items 104, etc.

In FIGS. 1 and 2A, the six UV light sources 108 of the UV lighting arrangement 104 are installed such that they emit UV lights downwards towards the open bottom of the housing 100 (and optionally the shelves). The configuration of the UV light sources 108 in FIG. 2A are shown to emit UV light perpendicular to an extension of the top section 115 and thus directly towards the open bottom portion. Here, the UV light from the respective UV light sources 108 are indicated as generated in a “cone-based””, where the UV light cones overlap a predefined distance, D, from the bottom of the housing 100, i.e. in FIG. 2A being the table 101. The predefined distance, D, may in some embodiments be e.g. 3-10 cm, such that items arranged at least at the start of the overlap is guaranteed to be receiving UV light from at least one UV light source 108.

Alternatively, as is shown in FIG. 2B, the UV light sources 108 may be arranged slightly angles as compared to the configuration as is presented in FIG. 2A. Here, the overlap will be three-dimensional, possibly ensuring that items 108 are ensured to receive UV light also at “sided” and not just from a top direction. Such an implementation may possibly be advantageous in case of the above-mentioned inclusion of one or a plurality of shelves within the housing 101.

In line with the present disclosure, it may be possible to adapt the comprises control unit 112 such that the UV light sources 108 emits “enough” UV light for minimizing minimize microorganisms at the items 104. In one embodiment, the activation of the control unit 112 is made dependent on when and for how long the door 113 has been opened. As an alternative or also, the UV light sources 108 may be specifically activated once the control unit 112 has received an indication that the housing 101 has been “filled” with new items.

In a preferred embodiment of the present disclosure the system 10 further comprises a sensor 116 arranged to detect human activity within the housing 101 and preferably how a human is interacting with the items 104. Such a sensor may for example be selected from a group comprising a switch, a PIR sensor, a radar sensor, a reed switch, an opto electronic sensor, a pressure sensor, an image sensor. It may in some embodiments be preferred to make use of a sensor 116 that is arranged to determine where the human is interacting with the items 108. The control unit 112 may then determine a time of interaction with the items 108 defined an activation time/UV dosage to be delivered by the UV light sources 108.

As suggested above, it is generally desirable to allow the UV lighting arrangement 102 to comprise a plurality of UV light sources 108 in order to ensure that all of the plurality of items 104 are covered with UV radiation. Additionally, it should also be noted that certain microorganism may be required to receive the above-mentioned dose of UV light/UVC irradiation in order to deactivate the microorganisms on the surface to a specified level.

This dose D may be expressed as:

$$D = I \times t$$

where I is the intensity, e.g. expressed in mW/cm² from the light sources of the UV lighting arrangement 104 onto the items 104, and t is the time during which the irradiation is applied.

The intensity I on the surface may then possibly be expressed as:

$$I = I ( o ) / A$$

where I(o) is the intensity as zero distance from the light sources and A is the area on surface 102 on which the radiation is to be distributed.

This area may in turn be expressed as:

$$π \times {r}^{2}$$

where r is the radius and is expressed as r=d×tan(v), where d is the distance between the surface and the UV source, and v is the cone angle as defined above.

In this slightly simplified example, the intensity is assumed constant over the beam angle and absorption in the media between the UV light sources and the surface 102 are neglected. More accurate calculations are entirely feasible and straight forward but are not deemed necessary in this description.

Now it is easy to realize that an increased distance may yield a larger area covered onto the items 104, by a single UV light source (i.e. in an embodiment where the UV lighting arrangement 104 only comprises a single UV light source). This will however also result in a lower intensity I on this surface and the time to reach the required does D will be correspondingly increased.

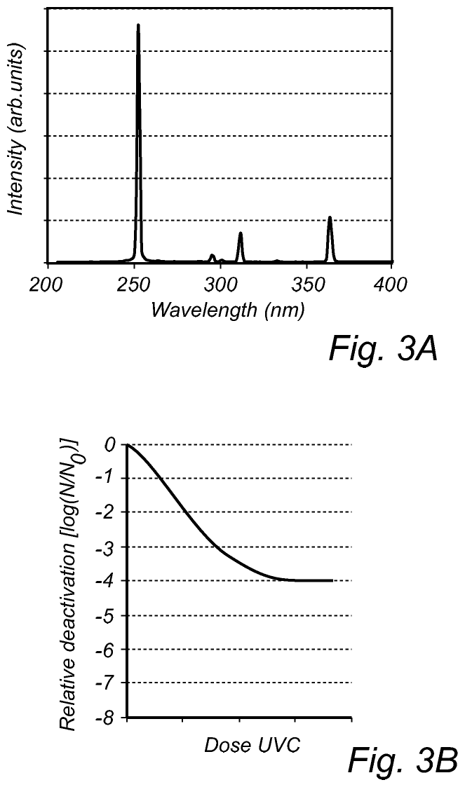

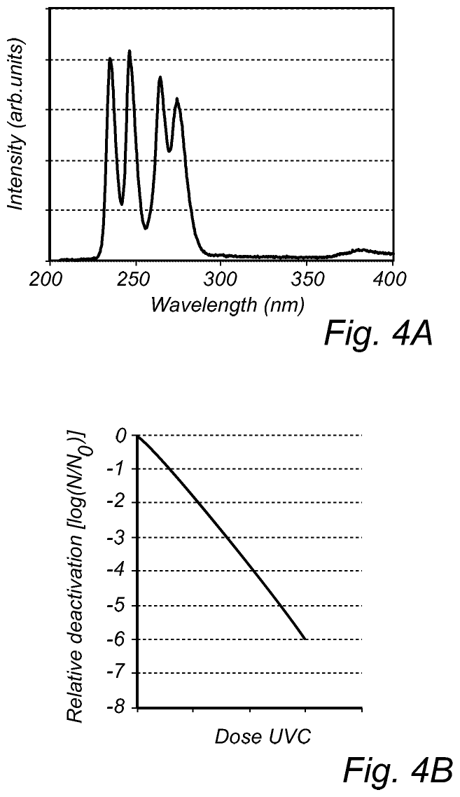

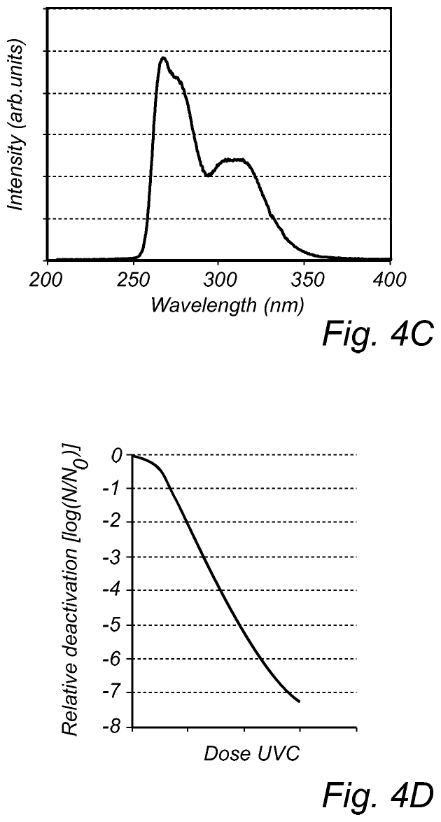

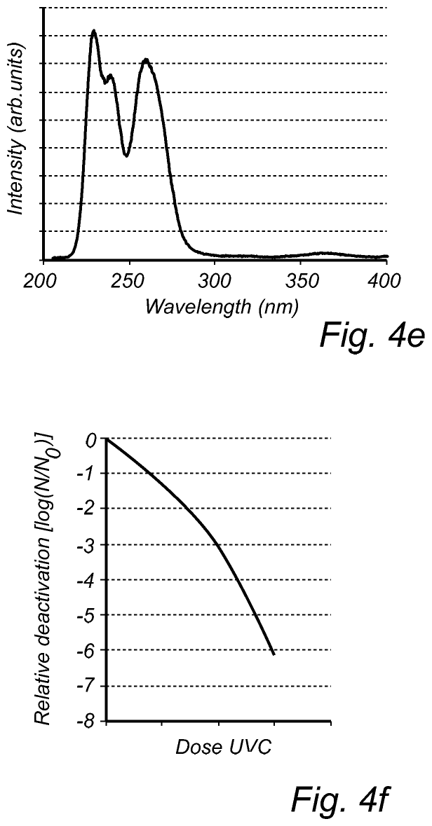

Turning now to FIGS. 3A and 3B, 4A-4F. Note that all measured de-activation curves show the relative reduction as function of UV dose in order to be comparable, thus the vertical axis shows the logarithm of the ratio between the remaining concentration of E. coli in Colony Forming Units per milliliter (CFU/ml)—denoted N—the initial concentration before irradiation, denoted No, thus denoted log(N/No).

As can be seen in FIG. 3A, a LP-Hg lamp essentially emits a strong relatively sharp peak at around 254 nm. FIG. 3B shows the corresponding deactivation of Escherichia coli (E. coli) at a surface. As can be seen, a certain level of E. coli is reached after which no further reduction is seen, i.e. the curve flattens over time at a set level.

Turning now to FIGS. 4A-4F, providing examples of results of use of the exemplary disinfection system shown in FIGS. 1A and 1B for de-activation of E. coli, where UV light is emitted within a wavelength range extending between at least 250 nm-310 nm. Note that all measured de-activation curves show the relative reduction as function of UV dose in order to be comparable, thus the vertical axis shows the logarithm of the ratio between the remaining concentration of E. coli in Colony Forming Units per milliliter (CFU/ml)—denoted N—the initial concentration before irradiation, denoted No, thus denoted log(N/No).

In FIG. 4A, the emission spectra from an UVC field emission light source provided with a first phosphor material (light powder) for UV light emission is provided. In FIG. 4A, the phosphor material has been selected to be a LuPO₄:Pr⁴⁺ phosphor material (or equivalent). In FIG. 4B, the corresponding de-activation curve is shown, for disinfection of water, where no significant tailing is visible.

In FIG. 4C, a second phosphor material in the form of a Lu₂Si₂O₇:Pr⁴⁺ phosphor material is used, and FIG. 4D shows the corresponding de-activation curve. As may be seen, in FIG. 4D, a de-activation of almost 8 orders of magnitude has been achieved, i.e. 99.9999999% of the bacteria have been de-activated.

Turning to FIGS. 4E and 4F, where a third phosphor material in the form of a LaPO₄:Pr⁴⁺ phosphor material is used and the corresponding de-activation curve is shown, respectively. The further disclosed electron-excitable UV-emitting material YBO₄:Pr⁴⁺ and YPO₄:Bi⁴⁺ provides similar results as shown in FIGS. 4A-4F.

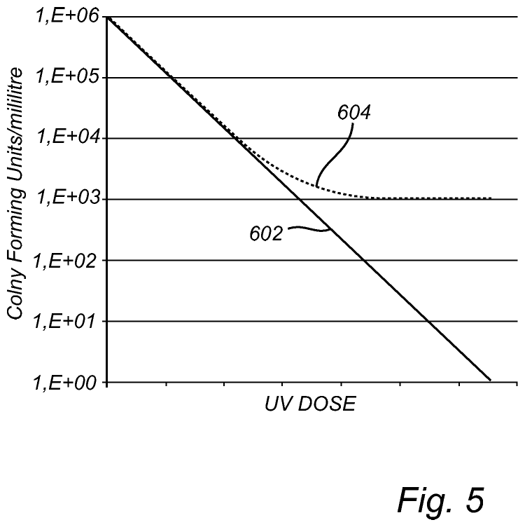

Turning to FIG. 5, which illustrates a mathematical model is used to describe the effect of inhibiting re-activation (and subsequent regrowth). In principle, the deactivation probability and the re-activation probability are set fixed. In this example the re-activation probability is 0.001 times the de-activation probability, an arbitrarily set number, but which gives results close to measured data. It is further postulated that the UV light can only deactivate non-de-activated organisms, and that the re-activation can only take place in de-activated organisms. As can be seen from FIG. 5 this will give a steady state situation, and a tailing effect will occur.

Looking back at the achieved test results (shown in FIGS. 3B and 4B) it is evident that the behavior is well explained by this model. There may very well be a second effect, such as a portion of microorganisms being more resistant to UV and requiring a higher dose of UV, but from these tests this second effect is at least not dominating, in fact must be much smaller than the re-activation effect. In FIG. 5 the reference 502 illustrates a mathematical modelling of the de-activation behavior with and a re-activation process and the reference 504 illustrates the mathematical modelling of the de-activation behavior without the re-activation process.

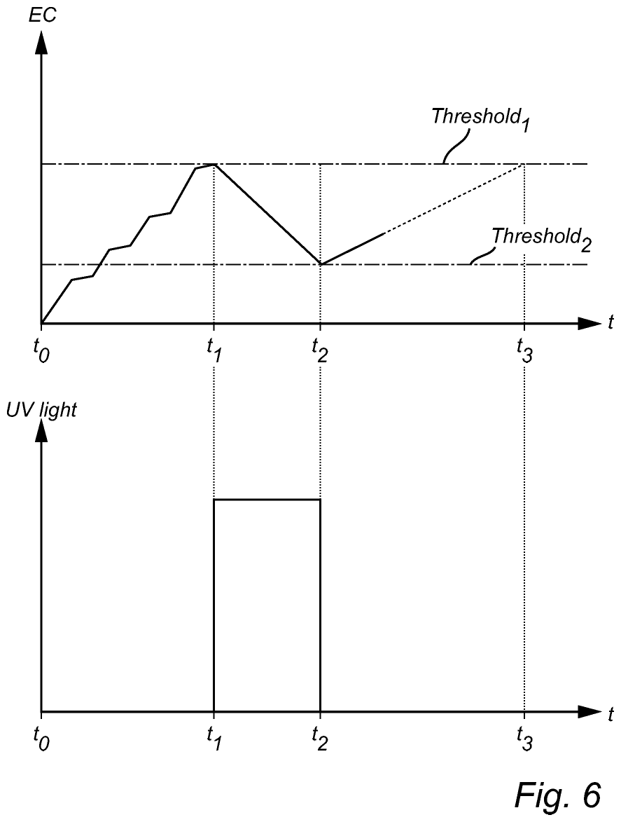

Turning finally to FIG. 6, presenting a relation between human activity within the housing 100 and the operation of the UV lighting arrangement 102. As has been discussed above, the general scheme according to the present disclosure is to ensure that the items within the housing are provided with an adequate amount of UV light to ensure that the items are safe to operate, as seen from a “microorganism perspective”. This is in accordance to the present disclosure achieved by making use of e.g. a sensor 116 for collecting information about human activity in relation to the items 104, whereby the control unit 112 may be used to estimate a human interaction with an interior of the housing 100 and to selectively activating the driver based on the estimated human interaction. Many different types of sensor 116 may be used for performing such an estimation. In the example below an image sensor is used, where the image sensor is arranged to acquire images of an interior of the housing 100. Preferably, the image sensor 116 (and related optics) is arranged at the top section 115 of the housing 100 and facing downwards towards the bottom of the housing 100. Thus, the image sensor 116 may acquire images covering the interior of the housing 100.

Based on image data collected by the image sensor 116 the control unit 112 may determine where and when human activity has been made. In the example below, the control unit 112 is arranged to estimate the human activity based on a “human contact area” and the time spent by the human in relation to said contact area. The control unit 112 will as such estimate a contact time×m² within the housing 100, below noted as K. Each human interaction may in turn be denoted as K₁, K₂, K₃, . . . , K_{N}, and the estimated accumulated interactions, K_{Acc}, will in turn be the sum of each human interaction, K_{Acc}=K₁+K₂+K₃+ . . . +K_{N}.

The estimated accumulated interactions, K_{Acc}, within the housing 100 will constitute on portion of an estimated contamination, EC, within the housing 100, including at the items 104. The estimated contamination, EC, may also be dependent on a temperature T within the interior of the housing 100 as well as an estimated microorganism growth MG over time. Not all temperatures are as problematic as seen from an estimated microorganism growth perspective. As an example, low temperatures (such as below 8° C.) as well as high temperatures (such as above 80° C.) will have an in comparison low impact on the estimated microorganism growth. This temperature dependency is below defined by the constant α, resulting in a temperature dependent estimated microorganism growth over time, denoted below as TαMGs.

The estimated contamination, EC, within the housing 100 will of course also be dependent on the dosage of UV lighting that is provided within the housing 100 over time, denoted below as UV_{Dosage} (e.g. in mJs/cm²), where UV light illumination of the interior of the housing 100 will reduce the estimated contamination, EC, within the housing 100.

Based on the above, the following equation may be defined:

$$EC = KAcc + T α MGs - {UV}_{Dosage}$$

The estimated contamination, EC, will in in turn be used by the control unit 112 for selectively activating the UV lighting arrangement 102. Preferably, the estimated contamination, EC is kept below a predefined first threshold, Threshold₁, thereby ensuring that the estimated contamination, EC, within the housing is kept at a desired level.

However, it is also preferred to introduce a predefined second threshold, Threshold₂, that is lower (closer to zero) as compared to the predefined first threshold, Threshold₁. The predefined second threshold, Threshold₂, will be used by the control unit 112 for determining when to deactivate the UV lighting arrangement 102. That is, it would according to the general scheme of the present disclosure be ineffective to control the UV lighting arrangement 102 until the estimated contamination, EC, within the housing 100 is at or close to zero.

As is illustrated in FIG. 6, the top diagram illustrates the estimated contamination, EC, within the interior of the housing 100. Between t₀ and t₁, the items 104 within the housing 100 are repeatedly interacted with by humans. As can be seen, the human interaction, K, as estimated by the control unit 112 based on the image data, is accumulated over time, resulting in the above mentioned estimated accumulated interactions, K_{Acc}. The estimated accumulated interactions, K_{Acc}, are in turn superimposed onto the temperature dependent estimated microorganism growth over time, TαMGs, as discussed above.

At t₁, the estimated contamination, EC, within the housing 100 is determined to be above the predefined first threshold, Threshold₁. Accordingly, as is illustrated in the bottom diagram the UV lighting arrangement 102 is activated and UV light is emitted towards the items 104. Between t₁ and t₂ the estimated contamination, EC, within the housing 100 is decreased based on the UV dosage generated by the UV lighting arrangement 102.

At t₂, the estimated contamination, EC, within the housing 100 has been reduced to the predefined second threshold, Threshold₂. Accordingly, the UV lighting arrangement 102 is, as shown in the bottom diagram, deactivated.

Between t₂ and t₃ no human interaction is identified within the housing 100, and thus the estimated contamination, EC, within the housing is only increased based on the temperature dependent estimated microorganism growth over time, TαMGs.

Further human interactions, K_{Acc}, and the general temperature dependent estimated microorganism growth over time, TαMGs, will of course result in further activations/deactivations of the UV lighting arrangement 102. Additionally, it may in some embodiments be advantageous to equip the system 10 with indicator means (such as e.g. a colored light source or similar) allowing an approaching human to be informed in case the estimated contamination, EC, within the housing 100 is above the first and/or the second predefined second threshold, Threshold₁/Threshold₂. In such a case, it may be suggested to not open the door 113 of the housing 100, since the items 104 at this point in time are determined to not be suitable for use since they are estimated to be “too contaminated”. In some embodiments the system 10 may be provided with a locking mechanism (not shown) provided to ensure that no humans are allowed to interact with the items 104/interior of the housing 100 in case the estimated contamination, EC, within the housing 100 is above the first and/or the second predefined second threshold, Threshold₁/Threshold₂.

It should be understood that the embodiment provided in relation to FIG. 6 is just an example of how the system according to the present disclosure may be operated. When other types of sensors (e.g. not necessarily being image sensors) are used for estimating the human activity, the control unit 112 may be adjusted to determine the contact time K. Similarly, in some embodiments it may be possible to disregard the temperature dependent estimated microorganism growth over time, TαMGs, and instead implement other means for simulating a general estimated growth within the housing as seen over time. Additionally, further factors may be taken into account for determining the estimated contamination, EC. Accordingly, it should be stressed that the general scope of protection is not limited to the embodiment provided in relation to FIG. 6.

In summary, the present disclosure relates to a portable system 10 for reducing microorganisms, wherein the system 10 comprises a housing 100 arranged to receive items 104 provided in relation to consumption of food, an ultraviolet (UV) lighting arrangement 102 positioned within the housing 100 and arranged to illuminate the items 104 received within the housing 100, a driver 110 adapted to provide a drive signal to the UV lighting arrangement 102, and a control unit 112 arranged to estimate a human interaction with an interior of the housing 100 and to selectively activating the driver 110 based on the estimated human interaction.

By means of the present disclosure, a system is provided that allows storage of items provided in relation to consumption of food, where the items are expected to be frequently and irregularly interacted with by humans, such as typically within a hospitality environment. The system has been specifically configured to estimate such a human interaction and control dosage of UV radiation based on the estimated human interaction. By means of such a system it is made possible to exactly control the UV radiation dose to limit spread and contamination of pathogens, while at the same time maximizing lifetime of the UV lighting arrangement. The resulting system will thus provide for a safer environment for the humans interacting with the stored items, at the same time as the cost of operation (of the system) is kept to a minimum.

Although the figures may show a specific order of method steps, the order of the steps may differ from what is depicted. In addition, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. Additionally, even though the invention has been described with reference to specific exemplifying embodiments thereof, many different alterations, modifications and the like will become apparent for those skilled in the art.

Variations to the disclosed embodiments can be understood and effected by the skilled addressee in practicing the claimed invention, from a study of the drawings, the disclosure, and the appended claims. Furthermore, in the claims, the word “comprising” does not exclude other elements or steps, and the indefinite article “a” or “an” does not exclude a plurality.

1. A portable system for reducing microorganisms, wherein the system comprises:
a housing arranged to receive items provided in relation to consumption of food,
an ultraviolet (UV) lighting arrangement positioned within the housing and arranged to illuminate the items received within the housing,
a driver adapted to provide a drive signal to the UV lighting arrangement, and
a control unit arranged to estimate an amount of human interaction with an interior of the housing and to selectively activating the driver based on the estimated amount of human interaction.

2. The system according to claim 1, wherein the housing comprises an openable portion for allowing the human interaction with the items.

3. The system according to claim 1, wherein the UV lighting arrangement comprises a plurality of UV light sources arranged spaced apart at a top section of the housing for achieving an UV light intensity uniformity at the items when the UV lighting arrangement is activated.

4. The system according to claim 1, further comprises at least one sensor arranged in communication with the control unit, wherein information from the at least one sensor is acquired by the control unit for estimating the human interaction.

5. The system according to claim 4, wherein the at least one sensor is selected from a group comprising a switch, a PIR sensor, a radar sensor, a reed switch, an opto electronic sensor, a pressure sensor.

6. The system according to claim 1, wherein the UV lighting arrangement comprises a plurality of non-mercury based UV light sources.

7. The system according to claim 6, wherein the plurality of non-mercury based UV light sources comprises at least one of field emission light sources and light emitting devices.

8. The system according to claim 2, wherein the openable portion is automatically opened when a human is detected in a vicinity of the system.

9. The system according to claim 1, wherein the control unit is further adapted to apply a predetermined minimum amount of UV radiation to the items over a predefined time period.

10. The system according to claim 1, wherein the housing comprises a plurality of side sections, and the side sections are arranged together to allow the housing to be foldable.

11. The system according to claim 1, wherein the housing has an open bottom section.

12. The system according to claim 1, wherein at least a portion of the housing is arranged to reflect UV light for containing emitted UV light within the housing.

13. The system according to claim 1, wherein the control unit is adapted to deactivate the UV lighting arrangement upon human interaction with the system.

14. The system according to claim 2, wherein the control unit is adapted to activate the UV lighting arrangement upon the openable portion of the housing being detected to be in a closed state.

15. The system according to claim 1, wherein the UV lighting arrangement is configured to emit UV light within a major portion of a wavelength range defined between 210-350 nm.

16. The system according to claim 7, wherein the plurality of field emission light sources comprises a light converting material arranged to receive electrons and to emit UV light.

17. The system according to claim 16, wherein the light converting material is selected to be at least one of LaPO4:Pr3+, LuPO3:Pr3+, Lu2Si2O7:Pr3+, YBO3:Pr3+, YAlO3:Sc3+ or YPO4:Bi3+ or a similar light converting material.

18. (canceled)

19. The system according to claim 1, further comprising a temperature sensor arranged within the housing, wherein the control unit is further adapted to selectively activating the driver based on an average temperature within the housing as determined over a predefined time period.

20. The system according to claim 1, further comprising at least one photovoltaic cell connected to the driver for delivering energy for powering the UV lighting arrangement.

21. The system according to claim 1, wherein the control unit is further adapted to selectively activating the driver based on at least one of a distance to the items, a target micro-organism, or an expected user behavior in relation to the items.
